(19) United States
(12) Patent Application Publication
(10) Pub. No.: US 2009/0124606 A1
(43) Pub. Date: May 14, 2009

(54) Composition for Treatment of Psychosis

(76) Inventors: Istvan Gacsalyi, Budapest (HU); Gyorgy Levay, Budakeszi (HU)

Correspondence Address: K.F. ROSS P.C., 5683 RIVERDALE AVENUE, SUITE 203 BOX 900, BRONX, NY, 10471-0900, US

(21) Appl. No.: 11/988,811

(22) PCT Filed: Jul. 12, 2006
(86) PCT No.: PCT/HU2006/000057

§ 371 Date: Jan. 14, 2008

(30) Foreign Application Priority Data
Jul. 14, 2005 (HU) P0500685

(51) Int. Cl. A61K 31/551; A61K 31/138; A61K 31/519; A61P 25/18; A61K 31/451

(52) U.S. Cl. 514/220; 514/646; 514/327; 514/259.41

(57) ABSTRACT

New pharmaceutical combination providing to decrease or eliminate the extrapyramidal side effects of antipsychotic active ingredients by combination of deramciclane with a classic antipsychotic agent (e.g. haloperidol, chloroprozamine or levoprozamin) or an atypical antipsychotic agent (e.g. risperidone, iloperidone or olanzapine).

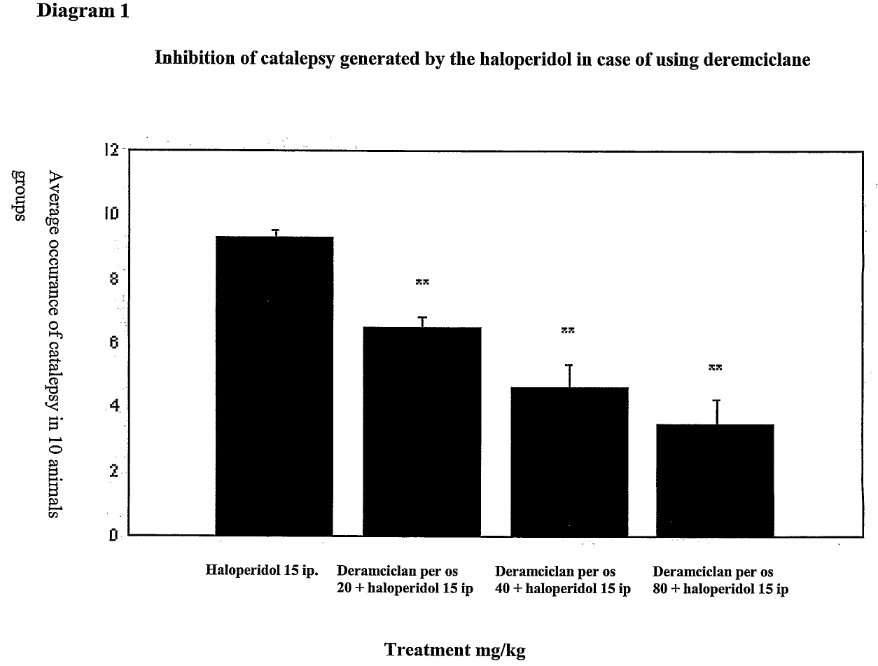

### FIELD OF THE INVENTION

The present invention relates to a pharmaceutical combination containing an antipsychotic compound as a first active pharmaceutical ingredient and (1R,2S,4R)-(−)-2-[N,N-(dimethylamino-ethoxy)]-2-phenyl-1,7,7-trimethyl-biciklo[2.2.1]heptane (INN name is deramciclane) of the formula

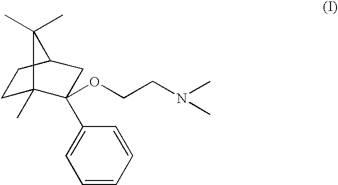

as the second active pharmaceutical ingredient. The antipsychotic active pharmaceutical ingredient can be a classical antipsychotic (e.g. haloperidol, chlorpromazine, levomepromazine etc.), or an atypical antipsychotic (e.g. risperidone, iloperidone, olanzapine etc.).

### TECHNICAL BACKGROUND OF THE INVENTION

During the administration of antipsychotics (neuroleptics) which compounds bind to the central D₂ receptors the extrapyramidal side effects have to be taken into consideration in case of short time administration and the evolution of tardive dyskinesia in case of longer administration period. (Ossowka K., Neuronal basis of neuroleptic-induced extrapyramidal side effects. Pol. J. Pharmacol., 2002 July-August, 54(4) 299-312).

These compounds cause catalepsy in animal tests. Symptoms of catalepsy are increased muscle-tone, rigidity and inactivity because of the inhibition of nigrostratal dopaminerg system.

Striatium and substantia nigra (nigrostratalis) are parts of the extrapyramidal system, therefore these symptoms can be considered as extrapyramidal symptoms. Such symptoms are usual side effects of the administration of haloperidol, risperidone, iloperidone, and similar compounds, because of these compounds bind to D₂ receptors.

There is a long felt need to reduce these side effects of antipsychotics for providing a possibility to increase their therapeutically used dose.

The aim described above reached surprisingly by co-administration of neuroleptic (antipsychotic) active pharmaceutical ingredients together with deramciclane.

Deramciclan does not cause catalepsy by itself however it binds also to central D₂ receptors similarly to neuroleptic agents. (Gacsályi et al, Receptor binding profile and anxiolytic activity of deramciclane (EGIS-3886) in animal models, Drug Dev. Res. 40: p. 333-348, 1997, Table 11.

TABLE 1
Bindings of Deramciclane on different receptors

| Receptor Affinity | (Ki nM/l) |
|---|---|
| Dopamine D₂ | 113.0 |
| 5-HT_{2A} | 11.0 |
| 5-HT_{2C} | 27.0 |

It is very surprising, that in the case of co-administration of deramciclane and neuroleptic agents, deranmciclane inhibits the cataleptic effect of the neuroleptic pharmaceutically active ingredients.

### SUMMARY OF THE INVENTION

The basis of the present invention is the surprising recognition that although deramciclane itself also binds to the central D₂ receptors, it is notwithstanding capable to reduce or eliminate of side effects caused by neuroleptics. These side effects are caused by receptor binding of these compounds to the central D₂ receptors.

The object of the present invention is a pharmaceutical composition containing an antipsychotic active ingredient or its pharmaceutically acceptable salt thereof and a compound of (1R,2S,4R)-(−)-2-[N,N-(dimethylamino-ethoxy)]-2-phenyl-1,7,7-trimethyl-bicyclo[2.2.1]heptane according to formula (I) or its pharmaceutically acceptable salt thereof.

### DETAILED DESCRIPTION OF THE INVENTION

More particularly, the present invention relates to a pharmaceutical composition which contains besides active ingredients comprising an antipsyhotic active pharmaceutical ingredient and deramciclane solid or fluid pharmaceutical carriers and/or auxiliary agents.

Chlorpromazine, levomepromazine, perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxyperline, molindone, sertindole, ziprasidone, flupenthixole, chlopenthixole, chrlorprothixene, tiotixene, zuclopenthixole, fluspirilene, pimozide, penfluridole, loxapine, clozapine, olanzapine, quetiapine, sulpiride, tiapride, amisulpiride, risperidone, or iloperidone, or their pharmaceutically accepted salts thereof can be used as antipsychotic agent.

In case of the use of haloperidol as antipsychotic active ingredient the pharmaceutical composition according to present invention contains 0.03-100 mg of deramciclane and 0.05-18 mg of haloperidol based on the dosage unit form, preferably 0.33-50 mg of deramciclane and 0.5-15 mg of haloperidol based on the dosage unit form, more preferably 0.67-10 mg of deramciclane and 0.75-7.5 mg of haloperidol based on dosage unit.

In case of the use of olanzapine as antipsychotic active ingredient, the pharmaceutical composition according to present invention contains 0.03-100 mg of deramciclane and 0.83-20 mg of olanzapine based on the dosage unit form, preferably 0.33-50 mg of deramciclane and 0.83-15 mg of olanzapine based on the dosage unit form, more preferably 0.67-10 mg of deramciclane and 1.67-10 mg of olanzapine based on the dosage unit form.

In case of the use of risperidone as antipsychotic active ingredient, the pharmaceutical composition according to the present invention contains 0.03-100 mg of deramciclane and 0.33-16 mg of risperidone based on the dosage unit form, preferably 0.33-50 mg of deramciclane and 0.67-12 mg of risperidone based on the dosage unit form, more preferably 0.67-10 mg of deramciclane and 0.67-8 mg of risperidone based on the dosage unit form.

Pharmaceutical compositions according to the present invention can contain also the antipsychotic active ingredients and deramciclane in form of pharmaceutically acceptable salts thereof in an amount which corresponds to the amount of bases described above.

Dearamciclan can be used as fumarate salt, (1R,2S,4R)-(−)-2-[N,N-(dimethylamino-ethoxy)]-2-phenyl-1,7,7-trimethyl-bicyclo[2.2.1]heptan-2-(E)-butenedioate (1:1) preferably.

Further object of the present invention is providing a process for the preparation of a pharmaceutical composition characterized in that an antipsychotic pharmaceutically active ingredient or therapeutically accepted salts thereof and (1R,2S,4R)-(−)-2-[N,N-(dimethylamino-ethoxy)]-2-phenyl-1,7,7-trimethyl-bicyclo[2.2.1]heptane or therapeutically accepted salts thereof are mixed with suitable solid or liquid carriers and/or auxiliary agents and converted to galenical form.

Further object of the present invention is the combined use of an antipsychotic drug and deramciclane together as pharmaceutically active ingredient. More particularly the use of an antipsychotic agent and deramciclane together for the preparation of an antipsychotic pharmaceutical composition, most particularly the use for the preparation of a pharmaceutical composition for treating schizophrenia.

The meaning of co-administration of deramciclane and an antipsychotic active pharmaceutical ingredient according to the present invention comprise cases in which the said compounds are in fix combination in a unit dosage form and both compounds are administered in the same time to the patient and the cases in which the active ingredients are subsequently administered.

Further object of the present invention is the use of an antipsychotic active ingredient or its pharmaceutically acceptable salts thereof and (1R,2S,4R)-(−)-2-[N,N-(dimethylamino-ethoxy)]-2-phenyl-1,7,7-trimethyl-bicyclo[2.2.1]heptane or therapeutically accepted salts thereof for the preparation of an antipsyhotic pharmaceutical composition.

Particularly, the object of the present invention is the use of an antipsychotic active ingredient or its pharmaceutically acceptable salts thereof and (1R,2S,4R)-(−)-2-[N,N-(dihmethylamino-ethoxy)]-2-phenyl-1,7,7-trimethyl-bicyclo[2.2.1]heptane or therapeutically accepted salts thereof for the preparation of a pharmaceutical composition for treating schizophrenia.

A further object of the present invention is the method of treatment in which an antipsychotic pharmaceutically active ingredient and deramciclane are co-administered in a pharmaceutically efficient amount to the patient who needs such treatment.

The compound (1R,2S,4R)-(−)-2-[N,N-(dimethylamino-ethoxy)]-2-phenyl-1,7,7-trimethyl-bicyclo[2.2.1]heptane (deramciclane) can be used in form of a pharmaceutically acceptable salt, most preferably as its fumarate salt, (1R,2S,4R)-(−)-2-[N,N-(dimethylamino-ethoxy)]-2-phenyl-1,7,7-trimethyl-bicyclo[2.2.1]heptan-2-(1)-butenedioate (1:1).

As it is known from the Hungarian Patent application No. P 99 01559, deramciclane can be prepared in high purity containing only a very small amount of (1R,3S,4R)-(−)-3-[2-N,N-(dimethylamino-ethyl)]-1,7,7,-trimethyl-bicyclo[2.2.1]heptan-2-on of the formula

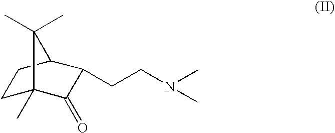

as contaminant.

Deramciclane used in the pharmaceutical compositions and in the course of the preparation of antipsychotic pharmaceutical compositions or pharmaceuticals compositions treating for schizophrenia and in the methods of treatment according to present invention contains preferably less than 0.2%, more preferably less than 0.05% of (1R,3S,4R)-(−)-3-[2-N,N-(dimethylamino-ethyl)]-1,7,7,-trimethyl-bicyclo[2.2.1]heptan-2-on of the formula (II) or corresponding acid additional salts thereof.

According to the present invention, antipsychotic active ingredients, antipsychotics (neuroleptics) are such compounds which are suitable for treating different psychotic disorders and/or diseases and bind to central D₂ receptors. Suitable compounds for example are as follows without limited the scope of all appropriate compounds to the content of the list: chlorpromazine, levomepromazine, perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxypertine, molindone, sertindole, ziprasidone, flupenthixole, chlopenthixole, chrlorprothixene, tiotixene, zuclopenthixole, fluspirilene, pimozide, penfluridole, loxapine, clozapine, olanzapine, quetiapine, sulpiride, tiapride, amisulpiride, risperidone, or iloperidone, or pharmaceutically acceptable salts thereof.

Pharmaceutically accepted salts according to the present invention are all salts of active ingredients with organic or inorganic acids which meet the requirements of pharmaceutical industry, (e.g. requirements of toxicity etc.).

The term psychosis is used as it is generally used in medicine. The psychosis is a symptomatic diagnosis. In the background of this diagnosis there can be some different disease which have different etiopatogenesis and outcome.

Diseases which are comprised in psychotic diseases for example are as follows without limited the scope to the content of the list: schizophrenia, schizoaffective illness, maniac depression disorder, moreover the organic psychiatric clinical pictures and psychotic status caused by toxic effects. Pharmaceutical compositions according to the present invention concern the treatment of such groups of diseases as described above. Daily dose (die) according to the present invention is such amount of the active ingredient, which is administered in a 24 hour period to the patient who needs it.

Dose range is the whole range of values of amount of active ingredients including the limiting values, which can be represented by doses of active ingredients during a 24 hour period of administration of the pharmaceutical composition (die).

Dosage unit forms according to the present invention are galenical forms e.g. tablets, injections or suppositories which contain an appropriate amount of active ingredients. Every pharmaceutical dosage forms which can be administered orally (e.g. powders tablets, film coated tablets, capsules, microcapsules, solutions, suspensions or emulsions) parenterally (e.g. intravenous, intramuscular, subcutan or intraperitonial injections or infusion compositions) or rectally (e.g. suppositories), transdermally (e.g. patches), or as an implant, or topically (e.g. creams, oiniments or patches) are galenical forms according to present invention.

Suitable carriers and auxiliary agents used in the pharmaceutical industry and the suitable processes for preparing pharmaceutical compositions are described in the literature (Remington's Pharmaceutical Sciences, Edition 18, Mack Publishing Co., Easton, USA, 1990).

Either solid or liquid pharmaceutical compositions according to the present invention can be prepared by known methods according to state of the art.

Solid pharmaceutical compositions according to the present invention can contain carriers and fillers (e.g. lactose, glucose, starch, calcium phosphate, microcrystalline cellulose), binding agents (e.g. gelatine, sorbitol, sodium carboximethyl-starch, crospovidone), disintegrating agents (e.g. croscaramellose, sodium-carboximethylcellulose, crospovidone), accessories used in processes of tablet preparation (e.g. magnesium stearate, talcum, polyethyleneglicol, silica or silicium dioxide) and tenzides (e.g. sodium laurylsuphate).

Liquid pharmaceutical compositions can be solutions, suspensions or emulsions and can contain suspending agents (e.g. gelatine, carboxymethylcellulose), emulsifiers (e.g. sorbitan monooleate), solvents (e.g. water, oils, glycerine, propylene-glycol, ethanol), buffer agents (acetate, phosphate, citrate buffers) and stabilizers (e.g. methyl-4-hydroxy-benzoate).

Liquid dosage forms acceptable for parenteral administration are aseptic isotonic solutions which can contain besides the solvents other auxiliary agents to control the pH and conserve the composition.

The active ingredients are homogenously dispersed in the carrier (e.g. in polyethyleneglycol or cocoa butter) of soft pharmaceutical compositions as suppositories.

Pharmaceutical compositions according to present invention can be prepared by processes known from the prior art using carriers, accessories and auxiliaries shown above or known from the pharmaceutical practice or literature.

Dose ranges of active ingredients according to the present invention in case of using haloperidol-deramciclane combination are as follows: 0.1-100 mg/die of deramciclane and 0.15-18 mg/die of haloperidol. Preferably, the dose ranges are 1-50 mg/die of deramciclane and 1.5-15 mg/die of haloperidol. Most preferably the dose ranges are 2-10 mg/die of deramciclane and 2.25-7.5 mg/die of haloperidol.

Dose ranges of active ingredients according to present invention in case of using olanzapine-deramciclane combination are as follows: 0.1-100 mg/die of deramciclane and 2.5-20 mg/die of olanzapine. Preferably, the dose ranges are 1-50 mg/die of deramciclane and 2.5-15 mg/die of olanzapine. Most preferably the dose ranges are 2-10 mg/die of deramciclane and 5-10 mg/die of olanzapine.

Dose ranges of the active ingredients according to the present invention in case of using risperidone-deramciclane combination are as follows: 0.1-100 mg/die of deramciclane and 1-16 mg/die of risperidone. Preferably, the dose ranges are 1-50 mg/die of deramciclane and 2-12 mg/die of risperidone. Most preferably the dose ranges are 2-10 mg/die of deramciclane and 2-8 mg/die of risperidone.

The adequate amounts of active ingredients in the pharmaceutical compositions or in the dosage units can be determined by person skilled in the art in case of known suitable daily doses and the chosen administration form.

Co-administration of deramciclane and antipsychotics are useful from the following point of views:

1. In case of increasing of the amount of used neuroleptics, the chance of extrapyramidal side effects are significantly lower. Due to this effect the therapeutic efficiency can be increased significantly.
2. The anti-anxiety effect which is a known feature of the majority of neuroleptic agents used in low dose, increases significantly in the combination therapy due to the synergistic effect of deramciclane.
3. The prevalence of the appearance of tardive dyskinesia as side effect significantly decreases or does not appear at all in case of long term administration of the combination.

The “complience”, the co-operation ability of patients increases as well significantly, because the use of a single pharmaceutical instead of two different. In case of elderly patients it is a frequent problem that the more pills are prescribed the more pills are misused.

In case of combination therapy the used dose rates are the same preferably which rates are used in case of monotherapy.

Present invention is shown more particularly in examples below without limiting of the scope of the protection to the examples. We prove in these examples that the deramciclane inhibits the extrapyramidal side effects caused by neuroleptics.

### EXAMPLE 1

### Inhibition of Catalepsy Generated by Haloperidol

Experiments were elaborated on 20-25 g weight NMRI mice. Groups of 10 mice were treated intraperitoneally with 15 mg/kg of haloperidol and carrier agent. Deramciclane (and carriers) were administered orally in different doses 60 minutes later. After a subsequent 60 minutes period mice were placed to a grid which has an inclination angle of 45°. In case of the animals were motionless for more than 30 seconds on the grid the events were seen as catalepsy. The procedure was repeated in every 30 minutes for 3 hours.

The effects are shown by percentage of changes compared to control group.

The results are shown in Table 2 and Diagram 1. It can be seen that deramciclane inhibits the catalepsy generated by haloperidol in a dose dependent way.

TABLE 2

| | Catalepsy |
|---|---|
| Administration (mg/kg) | (average ± SE)/10 animals |
| Haloperoidol 15 ip. | 9.3 ± 0.4 |
| Deramciclane 20 po. + haloperidol 15 ip. | 6.5 ± 0.7* |
| Deramciclane 40 po. + haloperidol 15 ip. | 4.7 ± 1.3* |
| Dearmciclane 80 po. + haloperidol 15 ip. | 3.5 ± 1.5* |

*= p < 0.05 compared to group treated with haloperidol
ip. = intraperitoneal administration
po. = per os administration

1. A pharmaceutical composition containing an antipsychotic agent or pharmaceutically acceptable salt thereof and (lR52̂,4R)-(−)-2-[N,N-(dimethylamino-ethoxy)]-2-phenyl-1,7,7-trimethyl-bicyclo[2.2.1]heptane of the formula 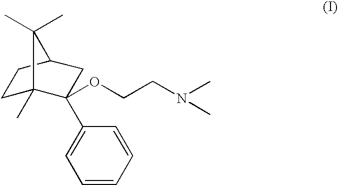
or therapeutically accepted salts thereof and inert pharmaceutically acceptable carrier(s) and/or accessories.

2. The pharmaceutical composition according to claim 1 wherein the used antipsychotic active ingredient is chlorpromazine, levomepromazine. perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxypertine, molindone, sertindole, ziprasidone, flupenthixole, chlopenthixole, chrlo?rothixene, tiotixene, zuclopenthixole, fluspirilene, pimozide, penfluridole, loxapine, clozapine, olanzapine, quetiapine, sulpiride, tiapride, amisulpiride, risperidone, or iloperidone, or pharmaceutically accepted salts thereof.

3. The pharmaceutical composition according to claim 1 haloperidol or its pharmaceutically acceptable salt are used as antipsychotic agent.

4. The pharmaceutical composition according to claim 3 wherein each unit dosage form of the pharmaceutical composition contains 0.03-100 mg of 1R,25′,4R)-(−)-2-[N,N-(dimethylamino-ethoxy)]-2-phenyl-1,7,7-trimethyl-bicyclo[2.2.1Jheptane or pharmaceutically accepted salts thereof, and 0.05-18 mg of haloperidol or pharmaceutically accepted salts thereof.

5. The pharmaceutical composition according to claim 3 wherein each unit dosage form of the pharmaceutical composition contains 0.03-50 mg of (1R,2£4R)-(−)-2-[N,N-(dimethylamino-ethoxy)]-2-phenyl-1,7,7-trimethyl-bicyclo[2.2.1]heptane or pharmaceutically accepted salts thereof, and 0.5-15 mg of haloperidol or pharmaceutically accepted salts thereof.

6. The pharmaceutical composition according to claim 3 wherein each unit dosage form of the pharmaceutical composition contains 0.67-10 mg of (1R,2S,4R)-(−)-2-[N,N-(dimethylamino-ethoxy)]-2-phenyl-1,7,7-trimethyl-bicyclo[2.2.1]heptane or pharmaceutically accepted salts thereof, and 0.75-7.5 mg of haloperidol or pharmaceutically accepted salts thereof.

7. The pharmaceutical composition according to claim 1 wherein olanzapine or pharmaceutically acceptable salt thereof are used as antipsychotic agent.

8. The pharmaceutical composition according to claim 7 wherein each unit dosage form of the pharmaceutical composition contains 0.03-100 mg of (1R,2,S,4R)-(−)-2-[N,N-(dimethylamino-ethoxy)]-2-phenyl-1,7,7-trimethyl-bicyclo[2.2.1]heptane or pharmaceutically accepted salts thereof, and 0.83-20 mg of olanzapine or pharmaceutically accepted salts thereof.

9. The pharmaceutical composition according to claim 7 wherein each unit dosage form of the pharmaceutical composition contains 0.33-50 mg of (1R,2S,4R)-(−)-2-[N,N-(dimemylamino-ethoxy)]-2-phenyl-1,7,7-trimethyl-bicyclo[2.2.1]heptane or pharmaceutically accepted salts thereof, and 0.83-15 mg of olanzapine or pharmaceutically accepted salts thereof.

10. The pharmaceutical composition according to claim 7 wherein each unit dosage form of the pharmaceutical composition contains 0.67-10 mg of (1R,2S,4R)-(−)-2-[N,N-(dimemylamino-ethoxy)]-2-phenyl-1,7,7-trimethyl-bicyclo[2.2.1]heptane or pharmaceutically accepted salts thereof, and 1.67-10 mg of olanzapine or pharmaceutically accepted salts thereof.

11. The pharmaceutical composition according to claim 1 wherein risperidone or its pharmaceutically acceptable salt are used as antipsychotic agent.

12. The pharmaceutical composition according to claim 11 wherein each unit dosage form of the pharmaceutical composition contains 0.03-100 mg of (1R,2S,4R)-(−)-2-[N,N-(dimethylamino-ethoxy)]-2-phenyl-1,7,7-trimethyl-bicyclo[2.2.1Jheptane or pharmaceutically accepted salts thereof, and 0.33-16 mg of risperidone or pharmaceutically accepted salts thereof.

13. The pharmaceutical composition according to claim 11 wherein each unit dosage form of the pharmaceutical composition contains 0.33-50 mg of (1R,2£,4R)-(−)-2-[N,N-(dimethylamino-ethoxy)]-2-phenyl-1,7,7-trimethyl-bicyclo[2.2.1]heptane or pharmaceutically accepted salts thereof, and 0.67-12 mg of risperidone or pharmaceutically accepted salts thereof.

14. The pharmaceutical composition according to claim 11 wherein each unit dosage form of the pharmaceutical composition contains 0.67-10 mg of (1R,2,S,4R)-(−)-2-[N,N-(dimethylamino-ethoxy)]-2-phenyl-1,7,7-trimethyl-bicyclo[2.2.1]heptane or pharmaceutically accepted salts thereof, and 0.67-8 mg of risperidone or pharmaceutically accepted salts thereof.

15. The pharmaceutical composition according to claim 1 wherein (1R,2S,4R)-(−)-2-[N,N-(dimethylamino-ethoxy)]-2-phenyl-1,7,7-trimethyl-bicyclo[2.2.1]heptan-2-(E)-butenedioate (1:1) is used as pharmaceutically acceptable salt of (1R,2S,4R)-(−)-2-[N,N-(dimethylamino-ethoxy)]-2-phenyl-1,7,7-trimethyl-bicyclo[2.2.1]heptane.

16. The pharmaceutical composition according to claim 1 wherein the used (li?,25′,4R)-(−)-2-[N,N-(dimethylamino-ethoxy)]-2-?henyl-1,7,7-trimethyl-bicyclo[2.2.1]heptane or its pharmaceutically acceptable salt contains less than 0.2%, preferably less than 0.05% of (1R,3S,4R)-(−)-3-[2-N,N-(dimethylamino-ethyl)]-1,7,7,-trimethyl-bicyclo[2.2.1]heptan-2-on of the formula 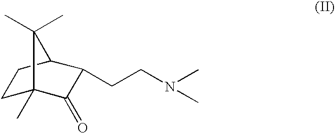
or pharmaceutically accepted salts thereof.

17. A process for preparation of a pharmaceutical composition according to claim 1 wherein the antipsychotic active ingredient or pharmaceutically acceptable salt thereof, and the (1R,2S,4R)-(−)-2-[N5N-(dimethylamino-ethoxy)]-2-phenyl-1,7,7-trimethyl-bicyclo[2.2.1]heptane according to formula (I) or pharmaceutically acceptable salt thereof are admixed with solid or fluid inert pharmaceutical carriers and/or auxiliary agents and converted to a galenical form.

18. The process for the preparation of pharmaceutical composition according to the claim 17 wherein the used antipsychotic active ingredient is chlorpromazine, levomepromazine, perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxypertine, molindone, sertindole, ziprasidone, flupenthixole, chlopenthixole, chrlorprothixene, tiotixene, zuclopenthixole, fluspirilene, pimozide, penfluridole, loxapine, clozapine, olanzapine, quetiapine, sulpiride, tiapride, amisulpiride, risperidone, iloperidone, or pharmaceutically accepted salts thereof.

19. The process for the preparation of pharmaceutical composition according to claim 17 wherein (1R,2S,4R)-(−)-2-[N,N-(dimethylamino-ethoxy)]-2-phenyl-1,7,7-trimethyl-bicyclo[2.2.1]heptan-2-(E)-butenedioate (1:1) is used as a pharmaceutically acceptable salt of (1R,2S,4i?)-(−)-2-[N,N-(dimethylamino-ethoxy)]-2-phenyl-1,7,7-trimethyl-bicyclo[2.2.1]heptane.

20. The process for the preparation of pharmaceutical composition according to claim 17 wherein the used (1R,2S,4R)-(−)-2-[N,N-(dimethylamino-ethoxy)]-2-?henyl-1,7,7-trimethyl-bicyclo[2.2.1]heptane or its pharmaceutically acceptable salts thereof contain less than 0.2%, preferably less than 0.05% of (1R,3S,4R)-(−)-3-[2-N,N-(dimethylamino-ethyl)]-1,7,7,-trimethyl-bicyclo[2.2.1]heptan-2-on of the formula (II) or pharmaceutically accepted salts thereof.

21. The use of compound (1R,2S,4R)-(−)-2-[N,N-(dimethylamino-ethoxy)]-2-phenyl-1,7,7-trimethyl-bicyclo[2.2.1]heptane or pharmaceutically acceptable salts thereof and an antipsychotic active pharmaceutical ingredient or pharmaceutically acceptable salts thereof in the manufacture of a medicament for the treatment of antipsychotic disorders.

22. The use of compound (1R,2S,4R)-(−)-2-[N,N-(dimethylamino-ethoxy)]-2-phenyl-1,7,7-trimethyl-bicyclo[2.2.1]heptane or pharmaceutically acceptable salts thereof and an antipsychotic active pharmaceutical ingredient or pharmaceutically acceptable salts thereof in the manufacture of a medicament for the treatment of schizophrenia.

23. The process for the preparation of pharmaceutical composition according claim 21 wherein the used antipsychotic agent is chlorpromazine, levomepromazine, perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxypertine, molindone, sertindole, ziprasidone, flupenthixole, chlopenthixole, chrlorprothixene, tiotixene, zuclopenthixole, fluspirilene, pimozide, penfluridole, loxapine, clozapine, olanzapine, quetiapine, sulpiride, tiapride, amisulpiride, risperidone, iloperidone, or pharmaceutically accepted salts thereof.

24. The process for the preparation of pharmaceutical composition according to claim 21 wherein (1R,2S,4R)-(−)-2-[N5N-(dimethylamino-ethoxy)]-2-phenyl-1,7,7-trimethyl-bicyclo[2.2.1]heptan-2-(E)-butenedioate (1:1) is used as a pharmaceutically acceptable salt of (1R,2S,4R)-(−)-2-[N,N-(dimethylamino-ethoxy)]-2-phenyl-1,7,7-a trimethyl-bicyclo[2.2.1]heptane.

25. The process for the preparation of pharmaceutical composition according to claim 21 wherein the used (1R,2S,4R)-(−)-2-[N,N-(dimethylamino-ethoxy)]-2-phenyl-1,7,7-trimethyl-bicyclo[2.2.1]heptane or its pharmaceutically acceptable salt contains less than 0.2%, preferably less than 0.05% of (1R,3S,4R)-(−)-3-[2-N,N-(dimethylamino-ethyl)]-1,7,7.-trimethyl-bicyclo[2.2.1]heptan-2-on of the formula (II) or pharmaceutically accepted salts thereof.

26. A method of treatment of psychotic diseases wherein (1R,2S,4R)-(−)-2-[N,N-(dimethylamino-ethoxy)]-2-phenyl-1,7,7-trimethyl-bicyclo[2.2.1]heptane or pharmaceutically acceptable salts thereof and an antipsychotic agent or pharmaceutically acceptable salts thereof are administered in a pharmaceutically efficient amount to the patient who needs it.

27. A method of treatment of schizophrenia wherein (1R,2S,4R)-(−)-2-[N3N-(dimethylamino-ethoxy)]-2-phenyl-1,737-trimethyl-bicyclo[2.2.1]heptane or pharmaceutically acceptable salts thereof and an antipsychotic agent or pharmaceutically acceptable salts thereof are administered in a pharmaceutically efficient amount to the patient who needs it.

28. The method of treatment according to claim 26 wherein the used antipsychotic agent is chlorpromazine, levomepromazine, perphenazine, pochlorperazine, thiopropazate, trifluoperazine, acetophenazine, thiproperazine, butaperazine, perazine, periciazine, thioridazine, mesoridazine, pipotiazine, haloperidol, trifluoperidol, melperone, moperone, pipamperone, bromperidol, benperidol, droperidole, fluanisone, oxypertine, molindone, sertindole, ziprasidone, flupenthixole, chlopenthixole, chrlorprothixene, tiotixene, zuclopenthixole, fluspirilene, pimozide, penfluridole, loxapine, clozapine, olanzapine, quetiapine, sulpiride, tiapride, amisulpiride, risperidone, iloperidone, or pharmaceutically accepted salts thereof.

29. The method of treatment according to claim 26 wherein 0.1-100 mg/die of deramciclane and 0.15-18 die mg/die of haloperidol, preferably 1-50 mg/die of deramciclane and 1.5-15 die mg/die of haloperidol, more preferably 2-10 mg/die of deramciclane and 2.25-7.5 die mg/die of haloperidol are administered.

30. The method of treatment according to claim 26 wherein 0.1-100 mg/die of deramciclane and 2.5-20 mg/die of olanzapine, preferably 1-50 mg/die of deramciclane and 2.5-15 die mg/die of olanzapine, more preferably 2-10 mg/die of deramciclane and 5-10 die mg/die of olanzapine are administered.

31. The method of treatment according to claim 26 wherein 0.1-100 mg/die of deramciclane and 1-16 mg/die of risperidone, preferably 1-50 mg/die of deramciclane and 2-12 die mg/die of risperidone, more preferably 2-10 mg/die of deramciclane and 2-8 die mg/die of risperidone are administered.

32. The method of treatment according to claim 29 wherein one or both active ingredients are administered in a sufficient amount in form of their pharmaceutically acceptable salts.

33. The method of treatment according to claim 26 wherein the (1R,2S,4R)-(−)-2-[N,N-(dimethylamino-ethoxy)]-2-phenyl-1,7,7-trimethyl-bicyclo[2.2.1]heptan-2-(E)-butenedioate (1:1) is used as a pharmaceutically acceptable salt of the compound of (1R,2S,4R)-(−)-2-[N,N-(dimethylamino-ethoxy)]-2-phenyl-1,7,7-trimethyl-bicyclo[2.2.1]heptane.

34. The method of treatment according to claim 26-33 wherein the used (1R,2S,4i?)-(−)-2-[N,N-(dimethylamino-ethoxy)]-2-phenyl-1,7,7-trimethyl-bicyclo[2.2.1]heptane or its pharmaceutically acceptable salts thereof contains less than 0.2%, preferably less than 0.05% of the compound of (1R,3S,4R)-(−)-3-[2-N.N-(dimethylamino-ethyl)]-1,7,7,-trimethyl bicyclo[2.2.1]heptan-2-on of the formula (II) or pharmaceutically accepted salts thereof.
